(19) United States
(12) Patent Application Publication
(10) Pub. No.: US 2008/0249591 A1
(43) Pub. Date: Oct. 9, 2008

(54) CONTROLLERS FOR IMPLANTABLE MEDICAL DEVICES, AND ASSOCIATED METHODS

(75) Inventors: Shan E. Gaw, Seattle, WA (US); Erin E. Mackintosh, Woodinville, WA (US); Matt L. Bielstein, Renton, WA (US); Don L. Carlson, Everett, WA (US)

Correspondence Address: PERKINS COIE LLP;PATENT-SEA, P.O. BOX 1247, SEATTLE, WA, 98111-1247, US

(73) Assignee: Northstar Neuroscience, Inc., Seattle, WA (US)

(21) Appl. No.: 11/697,703

(22) Filed: Apr. 6, 2007

(51) Int. Cl. A61N 1/00

(52) U.S. Cl. 607/60

(57) ABSTRACT

Controllers for implantable medical devices, and associated methods are disclosed. A device in accordance with one embodiment includes a hand-held housing, an image display device carried by the housing, and an input device carried by the housing. In particular embodiments, one portion of the housing can be rotatable relative to the other, or at a fixed, non-zero angle relative to the other, with the image display device and the input device carried by one portion and a wireless communication device carried by the other portion. A controller can be operatively coupled to the image display device to control the orientation of an image (e.g., whether the image is upright or inverted), presented at the display device in response to a signal received from the input device. The image can include a user-selectable icon, e.g., for controlling parameters associated with delivering therapeutic electromagnetic signals to a patient. Inverting the image can give both a patient and a practitioner improved access to the device.

### TECHNICAL FIELD

The present disclosure relates generally to controllers for implantable medical devices, including external hand-held controllers for implanted neurostimulators, and associated methods.

### BACKGROUND

Implantable medical devices (e.g., neurostimulators) are typically programmed at a physician's office or in a hospital setting. For example, a patient with an implantable, programmable device must typically go to a clinic to have a physician review the performance parameters of the device. Further, if the medical conditions of the patient warrant continuously monitoring or adjusting the device, the patient must remain under the physician's direct care for a period of time, which may be the duration of the treatment. Such a continual treatment plan imposes economic and social burdens on the patient and/or the physician. For example, as the number of implanted medical devices continues to increase, the result is a corollary increase in required resources at hospitals and clinics, thus escalating the overall cost of healthcare. In addition to the increase in cost and drain on resources, the patients are unduly restricted and inconvenienced by the need to either stay in the hospital, or make frequent visits to a clinic.

In response to this problem, various external programming devices have been developed for use with implantable medical devices. FIG. 1 illustrates one representative device. The device includes a portable programmer 20 having a display screen 22, a set of buttons 24, and a connector port 26. A cable 32 with a corresponding connector 30 couples the programmer 20 to a telemetry housing 36, which carries a telemetry coil 38. In various designs, the display screen 22 comprises a touch or pressure sensitive screen, and a significant amount of user input occurs via the touch screen (e.g., in response to user selections made with a input occurs via the touch screen (e.g., in response to user selections made with a stylus). Although portable, the device requires the patient to hold the telemetry housing 36 proximate to the implanted device (e.g., a pulse generator implanted beneath the patient's clavicle), while the portable programmer 20 is being programmed by the physician or operated by the patient, thus effectively requiring two sets of hands, or three hands, to operate. For instance, a patient may use one of their hands to maintain the telemetry housing 36 in a suitable position; while a medical professional uses one hand to hold the programmer 20 and another hand to communicate with the programmer 20 (e.g., through stylus-based selection of information presented upon the display screen 22). Alternatively, the telemetry housing 36 can be hung over the patient's shoulder while the programming device is operated; however, this leads to inefficiencies in the use of the device. Accordingly, there remains a need for a remote programming device that can be conveniently operated by the physician or the patient.

### BRIEF DESCRIPTION OF THE DRAWINGS

FIG. 1 is an isometric view of an external programmer configured in accordance with the prior art.

FIG. 2 is a top view of a patient and practitioner using a device configured in accordance with an embodiment of the invention.

FIG. 3 is an isometric view of an embodiment of the device shown in FIG. 2.

FIG. 4 is an isometric view of an embodiment of the device shown in FIG. 3, in a closed position

FIG. 5 illustrates information displayed by a device in accordance with an embodiment of the invention.

FIG. 6 is a side view of a device configured in accordance with an embodiment of the invention.

FIG. 7 is a top view of internal features of a device configured in accordance with an embodiment of the invention.

FIG. 8 is a flow chart illustrating a method in accordance with an embodiment of the invention.

FIG. 9 is an isometric view of a device having multiple display portions in accordance with an embodiment of the invention.

FIG. 10 is an isometric view of a device having rotatable display portions in accordance with an embodiment of the invention.

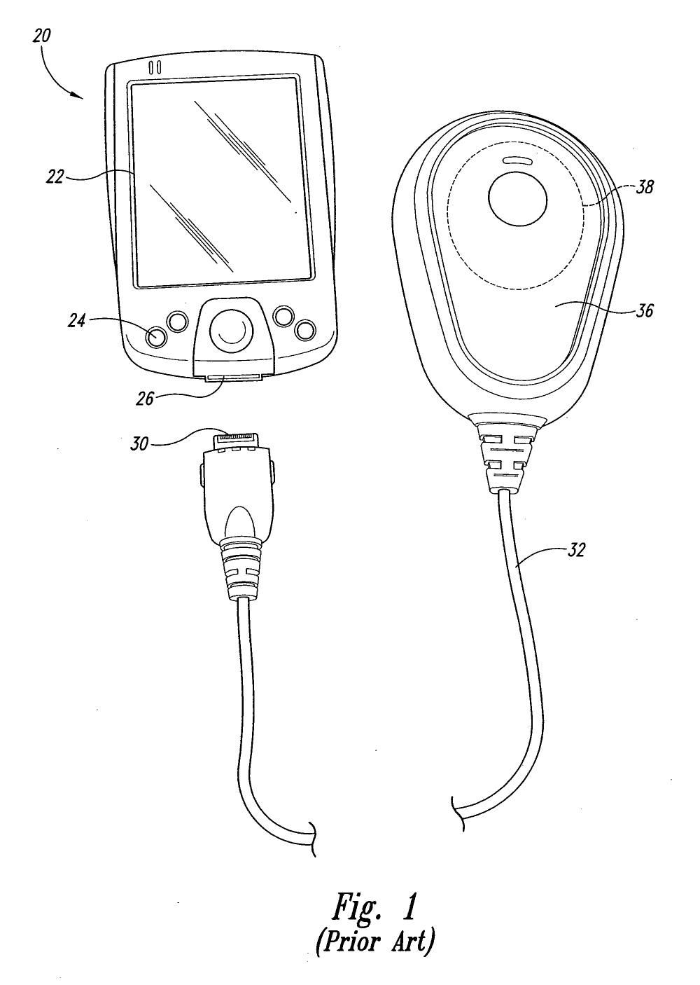

### DETAILED DESCRIPTION

The following disclosure describes several controllers and associated methods for remotely controlling implantable medical devices and/or other remotely controlled devices. The devices are generally arranged to enhance usability, whether the user is a practitioner or a patient. A patient therapy control device in accordance with one aspect includes a hand-held housing having a first portion and a second portion, with the first and second portions pivotable relative to each other, or fixed at a non-zero, non-normal tilt angle relative to each other. A wireless communication device can be carried by the first portion, and an input/output device can be carried by the second portion. The housing can further include a controller carried by at least one of the first and second portions. The controller can be coupled to the input/output device and the wireless communication device, and can be programmed with instructions for directing the delivery of therapeutic electromagnetic signals by an in-patient signal delivery device. The tilt feature of the control device can make it easier for the user to align the wireless communication device with the in-patient signal delivery device, while at the same time allowing the user easy access to the input/output device.

In other aspects, the patient therapy control device includes a hand-held housing, an image display device carried by the housing, and an input device. The patient therapy control device can further include a controller that is operatively coupled to the input device, and to the image display device to provide a display signal to the display device. The controller is programmed with instructions to control the orientation of an image, including a user-selectable icon, presented at the display device in response to a signal received from the input device. For example, the controller can selectively toggle the orientation of the image between a first orientation and a second orientation inverted (e.g., by 180°) relative to the first. This feature can make the device easy for both a patient and a practitioner to use, even if the patient and practitioner have opposite orientations relative to the device. In a further particular aspect, the controller is configured to operate in a first mode (e.g., a “patient” mode) with a first set of available instructions when the image has the first orientation relative to the display device. The controller is configured to operate in a second mode (e.g., a “practitioner” mode) with a second set of available instructions when the image has the second orientation, with the second set of instructions different than the first set.

A computer-readable medium in accordance with another aspect includes instructions for directing a user-selectable icon to have a first orientation relative to a display medium at which the icon is displayed. The user-selectable icon corresponds to a parameter with which electromagnetic signals are applied to a patient's central nervous system. The instructions can further include directing the user-selectable icon to invert from the first orientation to a second, inverted orientation in response to receiving a first input signal, and directing the user-selectable icon to revert to the first orientation in response to receiving a second input signal.

Aspects of the foregoing arrangements are expected to provide users (e.g., both practitioners and patients) with increased visual and manual access to the hand-held device while making adjustments to parameters in accordance with which electromagnetic signals are applied to the patient. As a result, the device is expected to be more efficient and/or more effective in use.

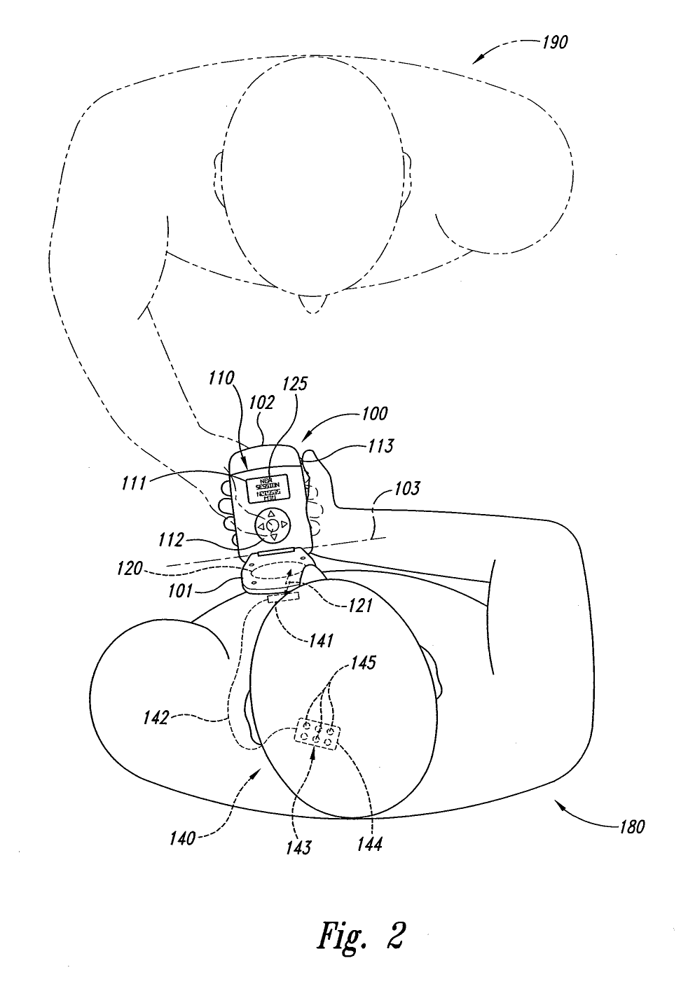

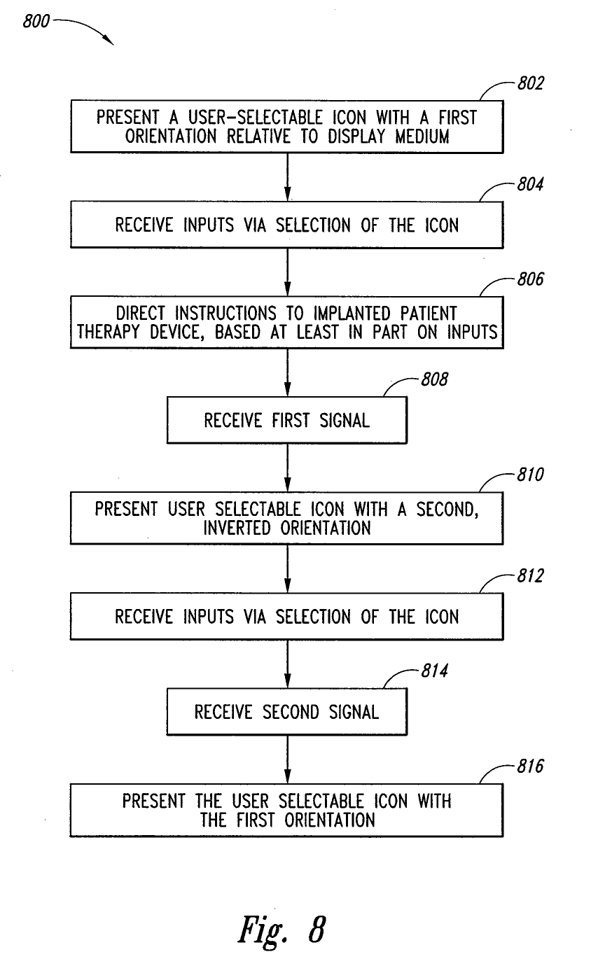

Specific details of certain embodiments of the invention are set forth in the following description and in FIGS. 2-10 to provide a thorough understanding of these embodiments to a person of ordinary skill in the art. More specifically, several systems in accordance with embodiments of the invention are initially described with reference to FIGS. 2-7, 9 and 10. A representative method is described with reference to FIG. 8. A person skilled in the relevant art will understand that the present invention may have additional embodiments, and that the invention can be practiced without several of the details described below.

External programmers, also known as downlink transmitters, can be used to transmit data to and receive data from implantable medical devices, also known as uplink transmitters. Examples of downlink transmitters include physician programmers, patient programmers, programming wands, telemetry access units, and the like. Particular aspects of the present invention include combining the physician and patient programmer functions into one hand-held external programmer device that can communicate with the implantable medical device, manage the patient's therapy, and/or collect implantable medical device data. Further particular aspects are directed to enhancing the functionality and usability of the device in light of its dual role.

FIG. 2 illustrates a patient 180 with an implanted patient device 140. The patient 180 is holding an external hand-held control device 100 that communicates with the implanted patient device 140 via a wireless communication link 121. A practitioner 190 is also shown in FIG. 2. Typically, either the patient 180 or the practitioner 190 holds the device 100 by him- or herself. However, for purposes of illustration, the practitioner 190 is shown (in phantom lines) holding the device 100 together with the patient 180. As discussed further below, aspects of the controls and displays carried by the device 100 allow the device 100 to be conveniently used by either the patient 180 or the practitioner 190, even though the patient 180 and the practitioner 190 have diametrically opposite positions relative to the hand-held device 100 and the implanted patient device 140. As is also discussed further below, either the patient 180 or the practitioner 190 can use the device 100 with only one hand, and so only the patient's and practitioner's right hands are shown in FIG. 2.

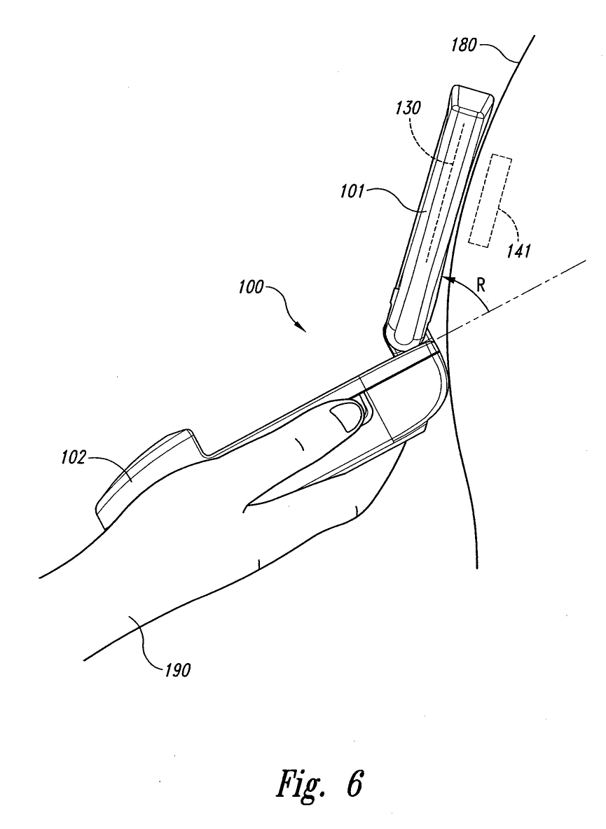

The hand-held device 100 can include a first portion 101 carrying a wireless communication device 120 that provides the wireless communication link 121. The device 100 can also include a second portion 102 carrying one or more input/output devices 110. The input/output devices 110 can include devices that only receive inputs, devices that only produce outputs, and devices that both receive inputs and produce outputs. The first portion 101 can have a tilted orientation relative to the second portion 102. In particular embodiments, the orientation can be adjusted by rotating the first portion 101 relative to the second portion 102 (or vice versa) about a rotation axis 103. This arrangement allows the user to orient the first portion 101 so that it readily communicates with the implanted patient device 140, while the second portion 102 is oriented so that the user (whether the patient 180 or the practitioner 190) has convenient visual and manual access to the input/output devices 110. For example, the patient 180 may wish to tilt the second portion 102 upwardly (as shown in FIG. 2) for more direct line-of-sight viewing. The practitioner 190 may wish to tilt the second portion 102 downwardly (as is discussed later with reference to FIG. 6) for the same reason.

In a particular embodiment, the implanted patient device 140 includes an implanted pulse generator 141 coupled to an electrode 143 with a lead 142. The electrode 143 can in turn include a support member 144 carrying one or more electrical contacts 145. In a further particular aspect, the electrode 143 is placed beneath or within the patient's skull, and the implanted pulse generator 141, which provides electrical pulses to the electrode 143, is placed below the patient's clavicle. Accordingly, when the hand-held device 100 is positioned to communicate with the implanted pulse generator 141, the first portion 101 can be rotated upwardly (e.g., out of the plane of FIG. 2) so as to rest against the patient's chest, therefore providing a robust wireless link 121 between the wireless communication device 120 and the implanted pulse generator 141. In other embodiments, the implanted patient device 140 may include a pulse generator 141 (or other device receiving wireless signals) that is implanted at a location other than a subclavicular location. In such instances, the tiltable first portion 101 can again be oriented appropriately so as to provide robust communication with the implanted device 140.

The illustrated second portion 102 includes a display 111 presenting one or more user-selectable icons 125, (a representative one of which is shown in FIG. 2) that guide the user as the user controls the instructions or other signals transmitted by the wireless link 121. As used herein, the term “icon” includes a wide variety of visual representations, e.g., text, symbols, and other graphical representations. The phrase “user selectable” indicates that the user can provide an input via interaction with the icon 125. For example, the user can highlight the icon 125 and “click” an input button or otherwise activate an input device. Alternatively, the user can align a curser with the icon and activate an input device. The display 111 can include a screen (e.g., an LCD screen) or other suitable device for presenting the icon 125 and other information to the user. The display 111 can be operatively coupled to a first input device 112 and a second input device 113, with the first input device 112 positioned for convenient manipulation by the practitioner 190, and the second input device 113 positioned for convenient manipulation by the patient 180.

The manner in which information is provided at the display 111 can be different depending upon whether inputs are received via the first input device 112 (e.g., from the practitioner 190) or the second input device 113 (e.g., from the patient 180). For example, the device 100 can be programmed with instructions for presenting the icon 125 with the orientation shown in solid lines in FIG. 2 when the patient 180 is providing inputs via the second input device 113. The icon 125 can have an inverted image (e.g., rotated 180°, as shown in phantom lines) with respect to the display 111 when the practitioner 180 is providing inputs via the first input device 112. Accordingly, the icon 125 and/or other information presented at the display 111 can be easily read by either the patient 180 or the practitioner 190, depending on whether the patient 180 is providing inputs or the practitioner 190 is providing inputs. Further details of specific features of the foregoing arrangement are described below.

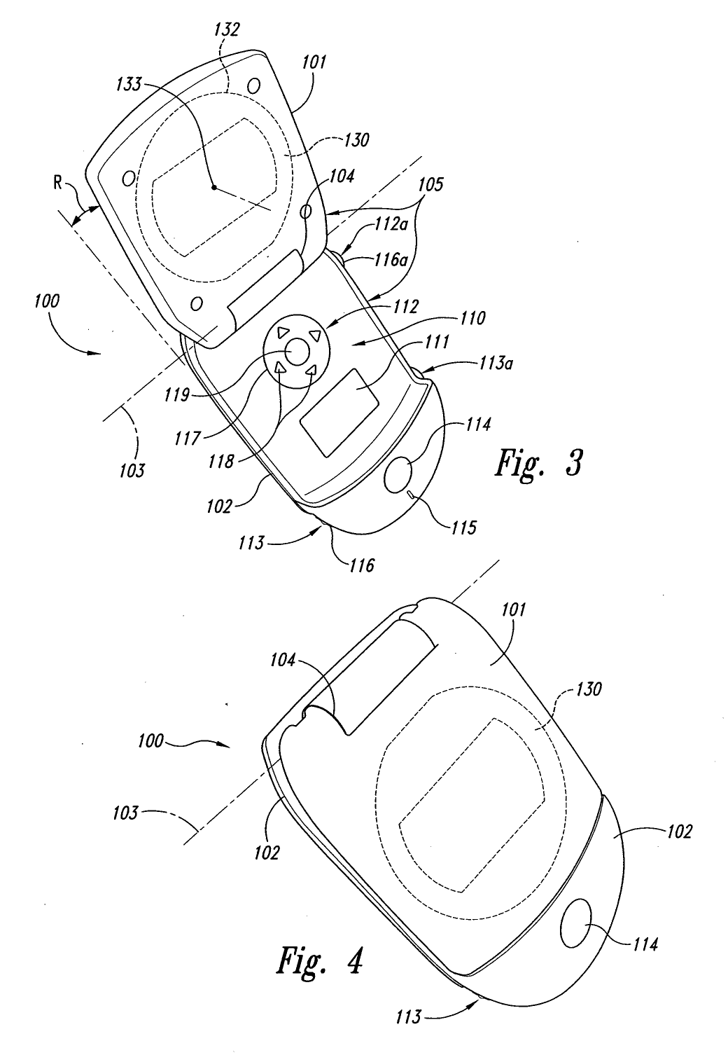

FIG. 3 is an enlarged view of the device 100 shown in FIG. 2. The device 100 includes a housing 105 which in turn includes the first portion 101 and the second portion 102 initially described above. The wireless communication device 130 carried by the first portion 101 can include an RF coil 132 (shown schematically in FIG. 3) arranged about a coil axis 133. In general, it may be desirable to have the coil axis 133 perpendicular to the patient's body while signals are communicated to the implanted patient device 140 (FIG. 2). Accordingly, the housing 105 at the first portion 101 can have a generally flat exterior surface that is generally parallel to the plane of the coil 132, and generally perpendicular to the coil axis 133.

The second portion 102 shown in FIG. 3 includes the display 111, the first input device 112, and the second input device 113, initially described above with reference to FIG. 2. The first input device 112 can include a navigation pad 117 that in turn includes four direction buttons 118 and a selection button 119. The user can move a cursor, highlighter or other visual cue up, down, left, and right over the display 111 with the direction buttons 118, and can make selections with the selection button 119. The second input device 113 can include a track wheel or thumbwheel 116 that allows the user to scroll through a menu by rolling the wheel clockwise and counterclockwise, and make a selection by pressing the wheel inwardly relative to the housing 105. If the menu presented at the display 111 includes selectable icons that may be accessed by left and right movement as well as by up and down movement, the cursor can scan through one line of menu options as the user rolls the track wheel 116, and then automatically jump to the next line and scan through it as the user continues to roll the track wheel 116 in the same direction.

In a particular embodiment, the central position of the first input device 112 allows it to be readily accessed by the practitioner's right hand or left hand. The location of the second input device 113 allows it to be readily accessed by the patient's right hand. In other embodiments, the input devices can have other arrangements. For purposes of illustration, other representative arrangements are shown in FIG. 3 superimposed on the arrangement discussed above, although a single device 100 may not include all such arrangements. For example, as shown in FIG. 3, the device 100 can include a different version of a second input device 113a, positioned on the opposite side of the second portion 102, to allow a left-handed patient easy access. In another embodiment (also shown in FIG. 3), the navigation pad 117 can be replaced with an alternatively-placed first input device 112a that includes a track wheel 116a positioned at an opposite corner of the second portion 102 relative to the second input device 113. Accordingly, the patient can access the second input device 113 with his or her right hand, and the practitioner can access the first input device 112a, also with his or her right hand.

In still another embodiment, the same input device can be used by both the patient and the practitioner. For example, the navigation pad 117 can be used by both the patient and the practitioner, and an additional provision can be made to determine whether it is the patient or the practitioner who is accessing the navigation pad 117 at any given time. One such provision includes configuring the selection button 119 to include a fingerprint sensor that scans the user's fingerprint. Based on the orientation of the fingerprint, the device 100 properly orients the images provided at the display 111. In another embodiment, the display 111 can provide both input and user orientation functions, in addition to the output functions described above. For example, the display 111 can include a touch-sensitive screen and a fingerprint scanner that determine the orientation of the user's finger, so as to orient the images provided at the display 111, either upright or inverted, depending upon the sensed orientation of the user's finger.

In any of the foregoing embodiments, the device 100 can include additional input/output devices 110, including a broadcast indicator 115 (e.g., an LED or other visual indicator) that notifies the user when a proper wireless link is established with the implanted patient device 140 (FIG. 2). The input/output devices 110 can also include a stop button 114 that immediately (or nearly immediately) terminates any stimulation signal or therapy output from the device 100 to the patient.

The first portion 101 and the second portion 102 can be rotatable relative to each other about the rotation axis 103 to provide the desired rotation angle R between these two components that allows the wireless communication device 130 to operate effectively, and also allows the user to have convenient visual and manual access to the input/output devices 110. Accordingly, the device 100 can include a hinge or other suitable rotatable coupling 104 connected between the first portion 101 and the second portion 102. An additional advantage of this feature is that it allows the device 100 to be folded closed when not in use. For example, FIG. 4 illustrates the device 100 in its closed position. In this position, the input/output devices are protected from the external environment. A latch or other suitable mechanism can be used to releasably secure the device 100 in the closed position. When the device 100 is opened, the user can release the second portion 102 while it remains in a desired position (e.g., with angle R at a value of between 0° and 180°), via the effect of friction at the rotatable coupling 104, or via springs, cams, detents or other suitable arrangements.

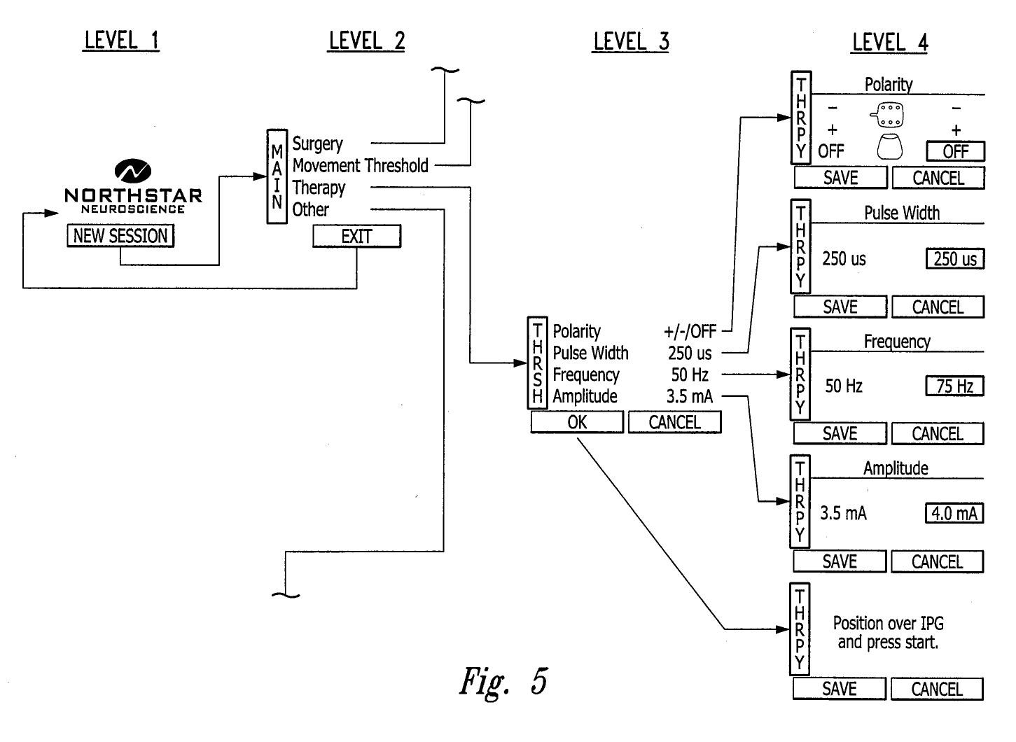

FIG. 5 illustrates representative menus that can be presented at the display 111 shown in FIG. 3. The menus have a sequentially presented, nested arrangement (indicated by Levels 1-4), and only representative submenus at Levels 3 and 4 are shown. Level 1 includes an introductory menu page at which a user can elect a new session by clicking on or otherwise activating a user-selectable “New Session” icon. Level 2 includes a main menu, illustrating option categories available to the user. These categories may include, for example, one or more of a “Surgery” category, a “Movement Threshold” category (for determining the patient's movement threshold), a “Therapy” category (for applying therapeutic stimulation to the patient), and an “Other” category for handling other functions. Level 3 functions are available for each of the Level 2 functions, but for purposes of illustration, only the functions associated with the therapy function of Level 2 are shown in FIG. 5. The Level 3 functions associated with therapy include selecting the polarity, pulse width, frequency, and/or amplitude of therapeutic electromagnetic signals applied to the patient. The Level 3 menu indicates the present value for each of these parameters. The Level 4 menus associated with each of these parameters are also shown in FIG. 5. Each Level 4 menu illustrates the present value for the parameter (shown without a surrounding box), and the selected new value for the parameter (shown with a surrounding box). Once the operator selects a new value, the operator can save the selection or cancel the selection, as desired.

In some cases, information presented at the multiple menu levels described above may be consolidated. For example, the device 100 can include multiple, pre-set, user-selectable mode packages, each of which has a pre-packaged set of values for each of the stimulation parameters. In such a case, the use of the device can be simplified in that the user need only select from among several existing combinations of parameters. An advantage of this arrangement is that it can simplify the use of the device. Conversely, an advantage of the arrangements described above with reference to FIG. 5 is that they allow the user more flexibility over parameter value selection.

In at least some instances, it may be desirable to give the practitioner and the patient control over different sets of stimulation parameters, and/or different value ranges for a given parameter. For example, it may be desirable to give the patient control over only a subset of the parameters that the practitioner can control. Accordingly, the device described above can be configured to present the patient with a reduced number of menu options, as compared with the menu options presented to the practitioner. In such an instance, the device must recognize whether it is in a “patient mode” or a “practitioner mode,” and present the appropriate menu listing. This determination can be made based upon whether the device is receiving input signals from a patient (e.g., received via the second input device 113 shown in FIG. 2) or from a practitioner (e.g., received via the first input device 112 shown in FIG. 2). The device may also include safeguards to prevent an unauthorized user from carrying out either patient-accessible functions or practitioner-accessible functions. For example, when the device receives an input signal from the first input device 112, it can request a practitioner-specific password before implementing any instructions. When the device receives an input via the second input device 113, it can request a patient-specific password before implementing any instructions. The passwords (or other security arrangement) can be configured so that the practitioner has access to the patient mode, but the patient does not have access to the practitioner mode.

The first and second input devices 112, 113 (FIG. 2) and associated software can be configured so that the device does not change from one mode to another unless the corresponding input device is activated for a minimum period of time (e.g., about three seconds). This arrangement can prevent an unplanned shift from one mode to another when an input device is inadvertently activated for a brief time. The device can also default to either the practitioner mode or the patient mode, depending (for example) on who is expected to be the most frequent user of the device.

Hand-held devices having features generally similar to those described above can be used to control various implanted medical devices, for example, implanted cortical electrodes (including, but not limited to, the electrodes shown in FIG. 2), sub-cortical or deep brain electrodes, cerebellar electrodes, spinal column electrodes, vagal nerve (or other cranial or peripheral nerve) electrodes, transcranial electrodes and/or transcranial magnetic stimulators. In particular embodiments, the applied stimulation can be used to enhance neuroplasticity effects, for example, in a manner disclosed in U.S. Pat. No. 7,010,351, assigned to the assignee of the present application and incorporated herein in its entirety by reference. In other embodiments, the device can be used to control electromagnetic signals applied to a patient for purposes in addition to or in lieu of enhancing neuroplasticity. In any of these embodiments, a wide variety of patient dysfunctions can be treated by such devices, including dysfunctions affecting the central nervous system and/or peripheral nerves.

FIG. 6 illustrates a side view of an embodiment of the device 100 positioned adjacent to the patient 180. In this instance, the practitioner 190 is carrying the device 100, with the first portion 101 oriented generally upwardly. Accordingly, the wireless communication device 130 is aligned with and proximate to the implanted pulse generator 141 for wireless communication. At the same time, the second portion 102, including its input/output devices 110 is oriented downwardly relative to the first portion 101 for convenient visual or manual access by the practitioner 190.

In an embodiment described above with reference to FIG. 3, the first portion 101 can be pivotable relative to the second portion 102 to change the rotation angle R. In another embodiment, the first portion 101 can be fixed relative to the second portion 102 and accordingly, the device 100 need not include a rotatable coupling 104 (FIG. 4). This arrangement can be simpler than the arrangement described above with reference to FIGS. 3 and 4 in that it includes fewer movable components. Conversely, the arrangement described above with reference to FIGS. 3 and 4 can allow the user (e.g., the patient or the practitioner) to adjust the angle R between the first portion 101 and the second portion 102 to best suit the user's need and physiognomy, and can also allow the device 100 to be folded closed for storage.

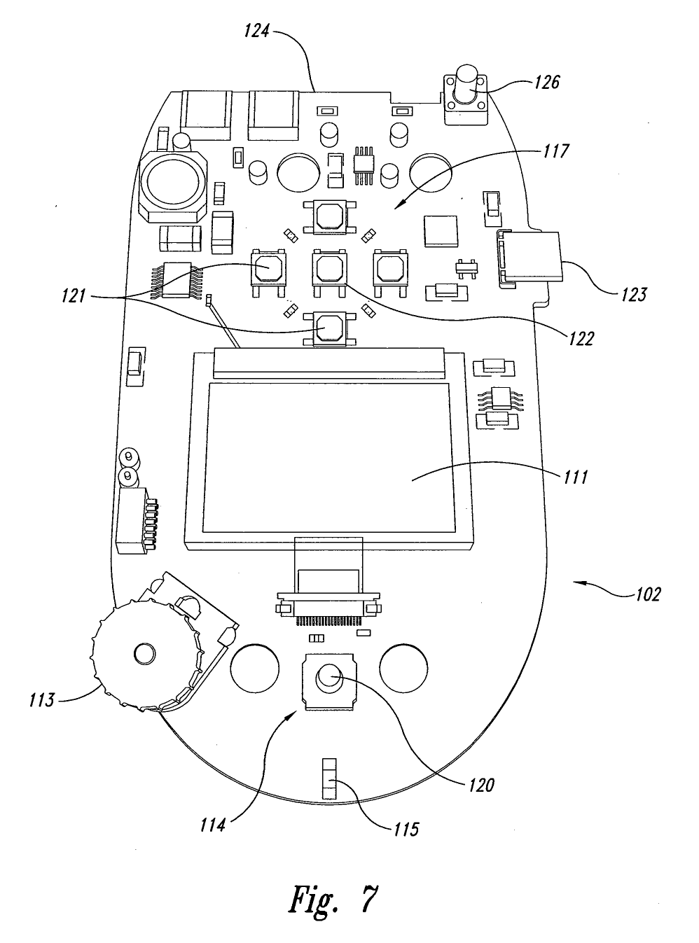

FIG. 7 illustrates representative internal components of the second portion 102, configured in accordance with an embodiment of the invention. The components can be carried by a printed circuit board 124 and can include the display panel 111, the broadcast indicator 115, a power switch 126, and a USB or other type of port 123 for communication with other devices. A power source (not visible in FIG. 7) is located on the back side of the printed circuit board 124. The internal components of the navigation pad 117 can include direction switches 121 and a selection switch 122. The internal components of the stop button 114 can include a stop switch 120. Additional components carried by the printed circuit board 124 can include a processor and/or other integrated circuit devices configured to receive inputs from the input devices and present information at the display 111.

FIG. 8 is a flow diagram illustrating a process for carrying out functions with devices, such as the devices 100 described above, in accordance with embodiments of the invention. The process 800 can include presenting a user-selectable icon with a first orientation relative to a display medium (process portion 802) and receiving inputs via selection of the icon while the icon has the first orientation (process portion 804). For example, process portion 804 can include receiving inputs from the patient via the second input device 113 shown in FIG. 2. Process portion 806 includes directing instructions to an implanted patient therapy device, based at least in part on the inputs. The instructions can include changing the parameter values in accordance with which electromagnetic signals are directed to the implanted patient device, activating the implanted patient device, and/or others.

In process portion 808, the process 800 includes receiving a first signal (e.g., via the first input device 112 shown in FIG. 2) and presenting the user selectable icon with a second, inverted orientation (process portion 810). For example, when a first signal corresponding to activation by the practitioner is received, the icon presented at the display inverts. In process portion 812, inputs are received via selection of the icon while the icon is in the second, inverted orientation. In process portion 814, a second signal is received (e.g. via the second input device 113 shown in FIG. 2). Based at least in part upon receipt of the second signal, the user selectable icon is presented at the first orientation (process portion 816), for example, to reorient the icon so as to appear upright to the patient.

One aspect of at least some of the foregoing embodiments is that the first and second portions of the hand-held device 100 are pivotable relative to each other. An advantage of this arrangement is that it allows the user, whether patient or practitioner, to orient the first portion 101 in a manner that facilitates communication between the wireless communication device and an implanted patient device, while also allowing the user to tilt the second portion 102 to an angle that facilitates visual access to the display 111 and manual access to the other input devices 110. This arrangement can make the device easier for both the patient and the practitioner to use.

Another feature of at least some of the foregoing embodiments is that the hand-held device 100 can either include a single input device that is accessible to both the patient and the practitioner, or multiple input devices, at least one of which is accessible to the patient, and another of which is accessible to the practitioner. This arrangement allows both the patient and the practitioner to use the device with relative ease. In addition, the hand-held device can automatically invert the orientation of images (e.g., menu pages and/or user-selectable icons) presented to the user, depending on whether the user is the patient or the practitioner. This feature can further enhance the usability and flexibility of the device.

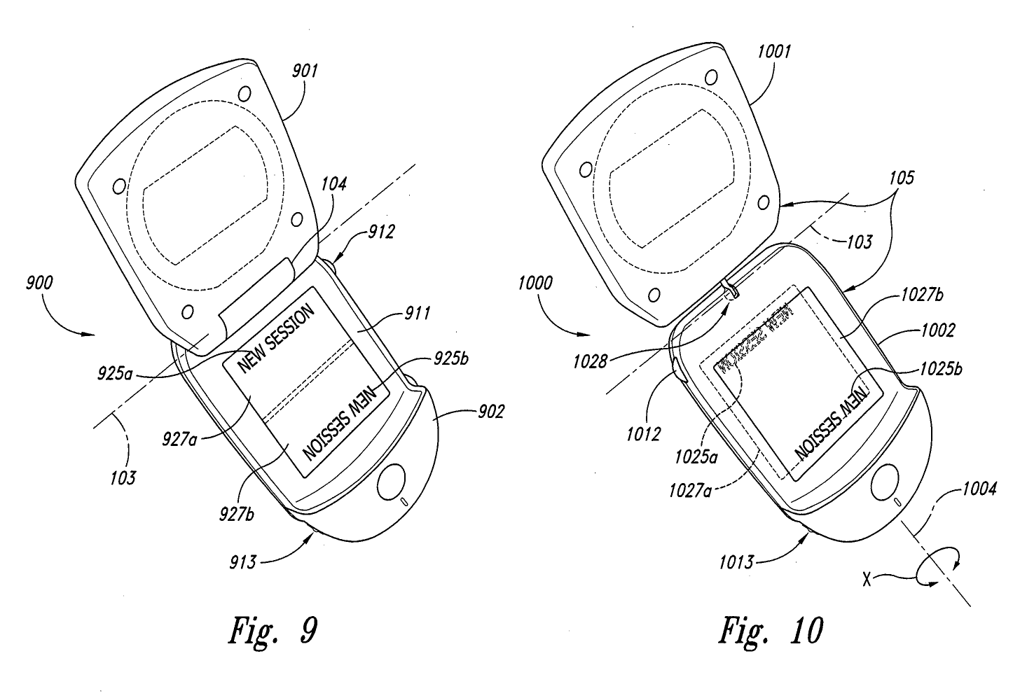

In other embodiments, hand-held devices can have other arrangements that also facilitate use by both a patient and a practitioner. For example, FIG. 9 illustrates a device 900 having a first portion 901 that is pivotable relative to a second portion 902 about the rotation axis 103. In this particular embodiment, the second portion 902 includes a display 911 that, in turn, has a first display portion 927a and a second display portion 927b. Each display portion 927a, 927b is configured to display a corresponding icon 925a, 925b, with the icons inverted relative to each other. Generally, only one of the icons 925a, 925b will be displayed at a given time, depending on which of two input devices is currently active. The input devices can include a first input device 912, generally activated by a practitioner, and a second input device 913 generally activated by the patient. The first and second display portions 927a, 927b can be sections of a single display screen, or they can be independently controllable screens, or they can have other arrangements. In any of these arrangements, each display portion 927a, 927b can support the display of an icon in an orientation that is inverted from the orientation presented by the other display portion.

FIG. 10 illustrates a device 1000 configured in accordance with still another embodiment. The device 1000 includes a first housing portion 1001 that is connected to a second housing portion 1002 with a ball and socket joint 1028, or other joint that supports rotation about multiple axes. Accordingly, the first and second housing portions 1001, 1002 can be folded relative to each other about the first axis 103 between an open configuration and a closed configuration. In addition, the second housing portion 1002 can be rotated relative to the first housing portion 1001 about a second axis 1004 that is generally transverse to the first axis 103, as indicated by arrow X. The second housing portion 1002 includes a first display portion 1027a and a second display portion 1027b that is located on the opposite side of the second housing portion 1002. Accordingly, in the orientation shown in FIG. 10, the first display portion 1027a faces downwardly into the plane of FIG. 10, and the second display portion 1027b faces upwardly out of the plane of FIG. 10. The second housing portion 1002 also includes a first input device 1012 and a second input device 1013. The second input device 1013 is accessible (e.g., by a right-handed patient) when the second housing portion 1002 has the orientation shown in FIG. 10. In this orientation, a second icon 1025b appears upright to the patient. When the second housing portion 1002 is rotated 180° about the second axis 1004, the first display portion 1027a faces outwardly, and the first input device 1012 is accessible to a right-handed practitioner holding the device in the manner shown in FIG. 2. The practitioner can then access the first input device 1012 to display and view a first selectable icon 1025a.

From the foregoing, it will be appreciated that specific embodiments of the invention have been described herein for purposes of illustration, but that various modifications may be made without deviating from the invention. For example, the input devices described above may have configurations other than those shown in the Figures. The information provided at the display may be presented and/or organized in manners other than those shown in the Figures. Aspects of the wireless communication links discussed above were described in the context of RF links, but can include other types of links (e.g., IR links) in other embodiments. Certain aspects of the invention described in the context of particular embodiments may be combined or eliminated in other embodiments. For example, an embodiment in which the first and second portions are fixed (as described above with reference to FIG. 6) can include other features generally similar to those described above with reference to FIGS. 3 and 4. Many of the features described above with reference to FIGS. 3-6 may be applied to embodiments shown in FIGS. 8 and 9. Such features include, for example, automated security features and automated techniques for determining which display portion is “active” based on which input device is transmitting input signals. Further, while advantages associated with certain embodiments of the invention have been described in the context of those embodiments, other embodiments may also exhibit such advantages, and not all embodiments need necessarily exhibit such advantages to fall within the scope of the invention. Accordingly, the invention is not limited except as by the appended claims.

I/we claim:

1. A patient therapy control device, comprising:
a hand-held housing having a first portion and a second portion, with the first and second portions positioned at or pivotable to a non-zero, non-normal tilt angle relative to each other;
a wireless communication device carried by the first portion;
an input/output device carried by the second portion; and
a controller carried by at least one of the first and second portions, the controller being coupled to the input/output device and the wireless communication device, the controller being programmed with instructions for directing the delivery of therapeutic electromagnetic signals by an in-patient signal delivery device.

2. The device of claim 1 whether the first and second portions are moveable relative to each other over a range of tilt angles.

3. The device of claim 1 wherein the first and second portions are fixed relative to each other at a fixed tilt angle.

4. The device of claim 1, further comprising the signal delivery device, and wherein the signal delivery device includes an implantable pulse generator and at least one electrode contact.

5. The device of claim 1 wherein the input/output device includes a user-manipulable input button.

6. The device of claim 1 wherein the input/output device includes a display screen.

7. The device of claim 1 wherein the input/output device is one of multiple input/output devices.

8. The device of claim 1 wherein the input/output device includes an image display device and an input device, and wherein the controller is programmed with instructions to control the orientation of an image, including a user-selectable icon, presented at the display device in response to a signal received from the input device.

9. The device of claim 1 wherein the wireless communication device includes a wireless communication coil that is coiled about an axis and positioned in a coil plane generally transverse to the axis, and wherein the first portion includes an external surface generally parallel to the coil plane and positioned to be placed adjacent to a patient's body.

10. A patient therapy control device, comprising:
a hand-held housing;
an image display device carried by the housing;
an input device; and
a controller operatively coupled to the image display device to provide a display signal to the image display device, the controller further being coupled to the input device and being programmed with instructions to control the orientation of an image, including a user-selectable icon, presented at the image display device in response to a signal received from the input device.

11. The device of claim 10 wherein the controller is configured to operate in a first mode with a first set of available instructions when the image has a first orientation relative to the image display device, and wherein the controller is configured to operate in a second mode with a second set of available instructions when the image has a second orientation inverted relative to the first orientation, the second set being different than the first set.

12. The device of claim 11 wherein the second set includes fewer instructions than the first set.

13. The device of claim 11 wherein the first and second modes require different security access codes for activation.

14. The device of claim 11 wherein the first mode is a practitioner-accessible mode and wherein the second mode is a patient-accessible mode.

15. The device of claim 11 wherein the second mode is accessible to a user operating in the first mode, and wherein the first mode is inaccessible to a user operating in the second mode.

16. The device of claim 10 wherein the image display device includes a touch screen, and wherein the input device forms a portion of the image display device.

17. The device of claim 10 wherein the input device includes a user-activatable switch.

18. The device of claim 10 wherein the input device includes a sensor positioned to identify the orientation of a user's finger contacting the input device and transmit a signal corresponding to the orientation.

19. The device of claim 10 wherein the input device is one of at least two input devices carried by the housing, each input device being operatively coupled to the controller, and wherein the controller is programmed to control the orientation of the icon differently depending on which input device it receives a signal from.

20. The device of claim 19 wherein the input devices include a first input device positioned at a first side of the housing and second input device positioned at a second side of the housing opposite the first side of the housing.

21. The device of claim 19 wherein the input devices include a first input device positioned at a first corner of the housing and second input device positioned at a second corner of the housing opposite the first corner of the housing.

22. The device of claim 10, further comprising a wireless communication coil carried by one of the first and second portions and coupled to the controller.

23. The device of claim 10 wherein the controller is programmed with instructions for directing the delivery of therapeutic electromagnetic signals from an in-patient signal delivery device.

24. The device of claim 10 wherein the hand-held housing includes a first portion and a second portion pivotable relative to the first portion, and wherein the image display device and the input device are carried by the second portion, and wherein the system further comprises a wireless communication device carried by the first portion, the wireless communication device being coupled to the controller to transmit instructions directing the delivery of therapeutic electromagnetic signals from an in-patient signal delivery device.

25. A patient therapy control device, comprising:
a hand-held housing;
an image display device carried by the housing, the image display device having a first display portion and a second display portion;
an input device; and
a controller operatively coupled to the image display device to provide a display signal to the image display device, the controller being programmed with instructions to present an image with a first orientation at the first display portion, and present the image with a second orientation inverted relative to the first orientation at the second display portion, the image including a user-selectable icon.

26. The device of claim 25 wherein the first and second display portions are portions of a single display screen.

27. The device of claim 25 wherein the first display portion includes a first display screen and the second display portion includes a second display screen.

28. The device of claim 25 wherein the first and second display portions are part of a single display screen.

29. The device of claim 25 wherein the first and second display portions face in generally the same direction.

30. The device of claim 25 wherein the first and second display portions face in opposite directions.

31. The device of claim 25 wherein the controller is programmed with instructions to present the image at one or the other of the first and second display portions based at least in part on instructions received via the input device.

32. The device of claim 25 wherein the hand-held housing includes a first housing portion and a second housing portion pivotable relative to the first housing portion, and wherein the image display device and the input device are carried by the second housing portion, and wherein the system further comprises a wireless communication device carried by the first housing portion, the wireless communication device being coupled to the controller to transmit instructions directing the delivery of therapeutic electromagnetic signals from an in-patient signal delivery device.

33. A computer-readable medium for controlling a patient therapy device, the computer-readable medium having instructions for:
directing a user-selectable icon to have a first orientation relative to a display medium at which the icon is displayed, the user-selectable icon corresponding to a parameter with which electromagnetic signals are applied to a patient's central nervous system;
directing the user-selectable icon to invert from a first orientation to a second, inverted orientation in response to receiving a first input signal; and
directing the user-selectable icon to revert to the first orientation in response to receiving a second input signal.

34. The computer-readable medium of claim 33, further having instructions for presenting the image in the first orientation when the input signal is received by a first input device and presenting the image in the second, inverted orientation when the input signal is received by a second input device.

35. The computer-readable medium of claim 33, further having instructions for directing signals to an implanted patient therapy device via a wireless communication device.

36. The computer-readable medium of claim 33, further having instructions for presenting a first set of available instructions when the image has the first orientation, and presenting a second set of available instructions when the image has a second orientation, the second set being different than the first set.

37. The computer-readable medium of claim 36 wherein the second set includes fewer instructions than the first set.

38. The computer-readable medium of claim 36, further having instructions for:
accepting a first security access code for presenting the first set of instructions; and
accepting a second, different, security access code for presenting the second set of instructions.

39. A method for operating a display medium for controlling a patient therapy device, the method comprising:
presenting a user-selectable icon with a first orientation relative to a display medium at which the icon is presented, the user-selectable icon corresponding to a parameter with which electromagnetic signals are applied to a patient's central nervous system;
receiving inputs via selection of the icon while the icon has the first orientation;
presenting the user-selectable icon with a second, inverted orientation in response to receiving a first signal;
receiving inputs via selection of the icon while the icon has the second orientation; and
presenting the user-selectable icon with the first orientation in response to receiving a second signal.

40. The method of claim 39 wherein receiving inputs includes receiving instructions for applying electromagnetic signals to an in-patient signal delivery device.

41. The method of claim 39 wherein receiving the first input signal includes receiving the first input signal from a user-activatable switch.

42. The method of claim 39 wherein receiving the first input signal includes receiving the first input signal from a sensor positioned to identify different orientations of a user's finger contacting the sensor.

43. The method of claim 39 wherein receiving the first input signal includes receiving the first input signal from either of at least two input devices carried by a housing that also carries the display device.

44. The method of claim 43, further comprising presenting the image in the first orientation when the input signal is received from a first input device and presenting the image in the second, inverted orientation when the input signal is received from a second input device.

45. The method of claim 39 wherein receiving inputs includes receiving inputs corresponding to instructions for directing electromagnetic signals to an implanted patient therapy device, and wherein the method further comprises conveying the instructions to the implanted patient therapy device via a wireless communication device.

46. The method of claim 39, further comprising presenting a first set of available instructions when the image has the first orientation, and presenting a second set of available instructions when the image has a second orientation, the second set being different than the first set.

47. The method of claim 46 wherein the second set includes fewer instructions than the first set.

48. The method of claim 46, further comprising:
accepting a first security access code for presenting the first set of instructions; and
accepting a second, different, security access code for presenting the second set of instructions.
